(19) United States
(12) Patent Application Publication
(10) Pub. No.: US 2022/0331318 A1
(43) Pub. Date: Oct. 20, 2022

(54) USE OF AN MDM2 INHIBITOR FOR THE TREATMENT OF MYELOFIBROSIS

(71) Applicant: Novartis AG, Basel (CH)

(72) Inventors: Asa Eliasson, Basel (CH); Nelson Guerreiro, Basel (CH); Christophe Meille, Basel (CH); Hans Menssen, Basel (CH); K Gary J Vanasse, Chestnut Hill, MA (US); Monika Wroclawska, Basel (CH)

(21) Appl. No.: 17/642,334

(22) PCT Filed: Sep. 14, 2020
(86) PCT No.: PCT/IB2020/058515

§ 371 Date: Mar. 11, 2022

Related U.S. Application Data
(60) Provisional application No. 62/900,931, filed on Sep. 16, 2019.

(51) Int. Cl. A61K 31/506; A61K 31/519; A61P 35/00

(52) U.S. Cl. CPC A61K 31/506; A61K 31/519; A61P 35/00

(57) ABSTRACT

The invention relates to the use of an MDM2 inhibitor in the treatment of myelofibrosis (MF). The invention also relates to a pharmaceutical combination comprising a) an MDM2 inhibitor and b) at least one further therapeutic agent, preferably ruxolitinib or a pharmaceutically acceptable salt thereof.

The present invention relates to uses of an MDM2 inhibitor and combinations thereof.

### FIELD OF THE INVENTION

The invention relates to the use of an MDM2 inhibitor, in the treatment of myelofibrosis (MF). The invention also relates to a pharmaceutical combination for the treatment of MF comprising a) an MDM2 inhibitor and b) at least one further therapeutic agent.

### BACKGROUND OF THE INVENTION

Myeloproliferative neoplasms (MPNs) are a unique and heterogeneous group of hemopathies characterized by proliferation and accumulation of mature myeloid cells, including myelofibrosis (MF), essential thrombocythemia (ET) and polycythemia vera (PV). Importantly, MF is the most severe form of Philadelphia chromosome-negative (i.e. BCR-ABL1-negative) myeloproliferative neoplasms, with a prevalence estimated to be 2.2 per 100,000 population. Myelofibrosis (MF) can present as a de novo disorder (PMF) or evolve from previous PV or ET (PPV-MF or PET-MF). The range of reported frequencies for post-PV MF are 4.9-6% at 10 years and 6-14% at 15 years, respectively, and 0.8-4.9% for post-ET MF at 10 years and 4-11% at 15 years, respectively (S Cerquozzi and A Tefferi, Blood Cancer Journal (2015) 5, e366).

Regardless of whether MF developed from PV, ET or as a primary disorder, it is characterized by a clonal stem cell proliferation associated with production of elevated levels of several inflammatory and proangiogenic cytokines resulting in a bone marrow stromal reaction that includes varying degrees of reticulin and/or collagen fibrosis, osteosclerosis and angiogenesis, some degree of megakaryocyte atypia and a peripheral blood smear showing a leukoerythroblastic pattern with varying degrees of circulating progenitor cells. The abnormal bone marrow milieu results in release of hematopoietic stem cells into the blood, extramedullary hematopoiesis, and organomegaly at these sites. Clinically, MF is characterized by progressive anemia, leukopenia or leukocytosis, thrombocytopenia or thrombocythemia and multi-organ extramedullary hematopoiesis, which most prominently involves the spleen leading to massive splenomegaly, severe constitutional symptoms, a hypermetabolic state, cachexia, and premature death.

A considerable number of cytokine and growth factor receptors utilize non-receptor tyrosine kinases, the Janus kinases (JAK), to transmit extracellular ligand binding into an intracellular response. For example, erythropoietin, thrombopoietin and granulocyte monocyte colony stimulating factor are all known to signal through receptors that utilize JAK2. JAK activate a number of downstream pathways implicated in proliferation and survival, including the STATs (signal transducers and activators of transcription), a family of important latent transcription factors.

Myelofibrosis is now known to be a clonal stem cell disease characterized by molecular (JAK2V617F, MPLW515L/K) and cytogenetic (13q-,20q-) markers (Pikman Y, Lee B H, Mercher T, et al. PLoS Med. 2006; 3(7):e270; Scott L M, Tong W, Levine R L, et al. N Engl J Med. 2007; 356:459-468). The JAK2V617F mutation has been identified in over 95% of patients with PV and approximately 50% of patients with ET and PMF. Furthermore, in a preclinical setting, animal studies have demonstrated that this mutation can lead to an MF-like syndrome. The JAK2V617F mutation alters the JAK2 tyrosine kinase making it constitutively active. As a result, polycythemia, thrombocythemia and leukocytosis can develop independently from growth factor regulation. Even in patients lacking a confirmed JAK2 mutation, the detection of STAT activation suggests dysregulated JAK activity. In fact, regardless of the mutational status of JAK2, the malignant cells appear to retain their responsiveness to JAK activating cytokines and/or growth factors; hence, they may benefit from JAK inhibition. Although several JAK inhibitors, including ruxolitinib (brand name Jakavi) have been approved for the treatment of MF, they have only demonstrated effect in treatment of symptoms. Progression of the disease is not halted and eventually patients may die prematurely.

Patients with MF have shortened survival (median survival is 6.5 years) and greatly compromised quality of life (QoL). Contributing factors for shortened survival include leukemic transformation and thrombohemorrhagic complications and for the compromised quality of life severe anemia (often requiring red blood cell (RBC) transfusions), symptomatic enlargement of the spleen and liver, substantial MF-associated symptoms burden (MF-SB), and cachexia (Tefferi and Barbui 2019).

The only potential curative treatment for MF is allogeneic hematopoietic stem cell transplantation (ASCT), for which the great majority of patients are ineligible. Therefore, treatment options remain primarily palliative and aimed at controlling disease symptoms, complications and improving the patient's QoL. The therapeutic landscape of MF has changed with the discovery of the V617F mutation of the Janus kinase JAK2 gene present in 60% of patients with PMF or PET-MF and in 95% of patients with PPV-MF, triggering the development of molecular targeted therapy for MF (Cervantes 2014). JAK play an important role in signal transduction following cytokine and growth factor binding to their receptors. Aberrant activation of JAK has been associated with increased malignant cell proliferation and survival (Valentino and Pierre 2006). JAK activate a number of downstream signaling pathways implicated in the proliferation and survival of malignant cells including members of the Signal Transducer and Activator of Transcriptions (STAT) family of transcription factors.

JAK inhibitors were developed to target JAK2 thereby inhibiting JAK signaling. Ruxolitinib, as all agents of this class, mainly inhibits dysregulated JAK-STAT signaling present in all MF patients irrespective of their JAK2 mutational status, but is not selective for the mutated JAK2, which explains its efficacy in both JAK2-positive and -negative MF. Ruxolitinib is highly effective in reducing the spleen size and controlling the symptoms of MF, with this resulting in a marked improvement in the patient's QoL (Cervantes et al 2016). Ruxolitinib is the only JAK inhibitor that has been granted a marketing authorization, as a single agent, for the treatment of patients with PMF, PPV-MF or PET-MF and for the treatment of patients with PV who are resistant to or intolerant to hydroxyurea. Ruxolitinib is the only approved pharmacological treatment for MF patients with splenomegaly and/or clinical symptoms and is considered standard of care (SoC). Although ruxolitinib has changed the treatment paradigm of MF patients, there is no clear indication of its disease-modifying effect (Cervantes 2014) and therapy-related anemia is often an anticipated downside (Naymagon and Mascarenhas 2017, Mead et al 2015).

There remains a high unmet medical need to finding new and efficacious treatment options for advancing the treatment of myelofibrosis.

### SUMMARY OF THE INVENTION

It is an object of the present invention to provide for a medicament for the treatment of myelofibrosis. The present invention is based on the inventors' surprising finding that an MDM2 inhibitor is useful in the treatment of myelofibrosis in a subject.

The present invention is also based on finding that, an MDM2 inhibitor in combination with at least one further therapeutic agent is useful in the treatment of myelofibrosis in a subject.

In an embodiment, the MDM2 inhibitor is selected from the group consisting of: a compound having the structure of Formula (A)

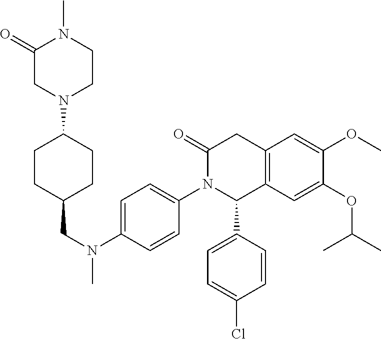 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

(A) (which is also known as CGM097) or a pharmaceutically acceptable salt thereof;

a compound having the structure of Formula (B)

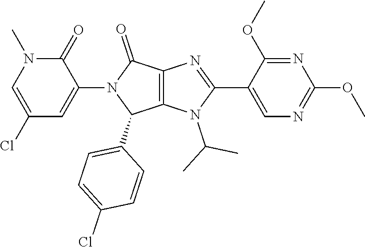 [SMILES: COc1ncc(-c2nc3c(n2C(C)C)[C@H](c2ccc(Cl)cc2)N(c2cc(Cl)cn(C)c2=O)C3=O)c(OC)n1]

(B) (which is also known as HDM201) and pharmaceutically acceptable salts thereof or a pharmaceutically acceptable non-covalent derivative (including salt, solvate, hydrate, complex, co-crystal) thereof.

Compounds (A) and (B) are also known as HDM2 (Human Double Minute-2) inhibitors.

In an embodiment, the MDM2 inhibitor is a compound having the structure of Formula (A), or a pharmaceutically acceptable salt thereof.

In another embodiment, the MDM2 inhibitor is a compound having the structure of Formula (B), or pharmaceutically acceptable salts thereof or a pharmaceutically acceptable non-covalent derivative (including salt, solvate, hydrate, complex, co-crystal) thereof.

The compound having the structure of Formula (B) is also known by its INN of siremadlin.

In an embodiment, the MDM2 inhibitor is in combination with a JAK inhibitor.

In an embodiment, the JAK inhibitor is a JAK1/2 inhibitor.

In an embodiment, the JAK inhibitor is ruxolitinib or pharmaceutically acceptable salt thereof.

In an embodiment, the MDM2 inhibitor and the JAK inhibitor are in the same formulation. In another embodiment, the MDM2 inhibitor and the JAK inhibitor are in separate formulations. In a further embodiment, the pharmaceutical combination is for simultaneous or sequential administration.

### DETAILED DESCRIPTION OF THE INVENTION

Certain terms used herein are described below. Compounds or biological agents of the present invention are described using standard nomenclature. Unless defined otherwise, all technical and scientific terms used herein have the same meaning as is commonly understood by one of skill in the art to which this invention belongs.

The term “combination,” “therapeutic combination,” or “pharmaceutical combination” as used herein refer to either a fixed combination in one dosage unit form, or non-fixed combination, or a kit of parts for the combined administration where two or more therapeutic agents may be administered independently, at the same time or separately within time intervals, especially where these time intervals allow that the combination partners show a cooperative, e.g., synergistic, effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

The term “MDM2 inhibitor” as used herein refers to a compound that selectively targets, decreases, or inhibits at least one activity of MDM2.

The term “JAK inhibitor” as used herein refers to a compound that selectively targets, decreases, or inhibits at least one activity of JAK.

The term “pharmaceutical composition” is defined herein to refer to a mixture or solution containing at least one therapeutic agent to be administered to a subject, e.g., a mammal or human, in order to prevent or treat a particular disease or condition affecting the mammal.

The term “pharmaceutically acceptable” as used herein refers to those compounds, biological agents (e.g., antibodies), materials, compositions and/or dosage forms, which are, within the scope of sound medical judgment, suitable for contact with the tissues of a warm-blooded animal, e.g., a mammal or human, without excessive toxicity, irritation, allergic response, and other problem complications commensurate with a reasonable benefit/risk ratio.

The terms “fixed combination,” “fixed dose,” and “single formulation” as used herein refers to a single carrier or vehicle or dosage form formulated to deliver an amount, which is jointly therapeutically effective for the treatment or prevention of cancer, of both therapeutic agents to a patient. The single vehicle is designed to deliver an amount of each of the agents, along with any pharmaceutically acceptable carriers or excipients. In some embodiments, the vehicle is a tablet, capsule, pill, or a patch. In other embodiments, the vehicle is a solution or a suspension.

The term “non-fixed combination,” “kit of parts,” and “separate formulations” means that at least one of the active ingredients, is administered to a patient as a separate entity either simultaneously, concurrently, or sequentially with no specific time limits, wherein such administration provides therapeutically effective levels of the two active ingredients agents in the body of the subject in need thereof. The latter also applies to cocktail therapy, e.g., the administration of three or more active ingredients.

The term “unit dose” is used herein to mean simultaneous administration of both agents together, in one dosage form, to the patient being treated. In some embodiments, the unit dose is a single formulation. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, injections, infusions, patches, or the like, administered to the patient at the same time.

An “oral dosage form” includes a unit dosage form prescribed or intended for oral administration.

The term “treating” or “treatment” as used herein comprises a treatment relieving, reducing, or alleviating at least one symptom in a subject or effecting a delay of progression of a disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease), and/or reduce the risk of developing or worsening a disease. The term “protect” is used herein to mean prevent, delay, or treat, or all, as appropriate, development, continuance or aggravation of a disease in a subject, e.g., a mammal or human. The term “prevent”, “preventing” or “prevention” as used herein comprises the prevention of at least one symptom associated with or caused by the state, disease or disorder being prevented.

The term “pharmaceutically effective amount,” “therapeutically effective amount,” or “clinically effective amount” of a combination of therapeutic agents is an amount sufficient to provide an observable or clinically significant improvement over the baseline clinically observable signs and symptoms of the disorders treated with the combination.

The term “jointly therapeutically active” or “joint therapeutic effect” as used herein means that the therapeutic agents can be given separately (in a chronologically staggered manner, especially a sequence-specific manner) in such time intervals that they prefer, in the warm-blooded animal, especially human, to be treated, still show an (preferably synergistic) interaction (joint therapeutic effect). Whether this is the case can, inter alia, be determined by following the blood levels of the compounds, showing that both compounds are present in the blood of the human to be treated at least during certain time intervals.

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from, at risk of suffering from, or potentially capable of suffering from cancers.

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, biological agents, salts, and the like, this is taken to mean also a single compound, salt, or the like.

The terms “about” or “approximately” are generally understood by persons knowledgeable in the relevant subject area, but in certain circumstances can mean within 20%, within 10%, or within 5% of a given value or range. Alternatively, especially in biological systems, the term “about” means within about a log (i.e., an order of magnitude) or within a factor of two of a given value.

Several MDM2 inhibitors are known to one of skill in the art and are within the scope of the combination of the invention.

In an embodiment, the MDM2 inhibitor is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, which is a compound having the structure of Formula (A).

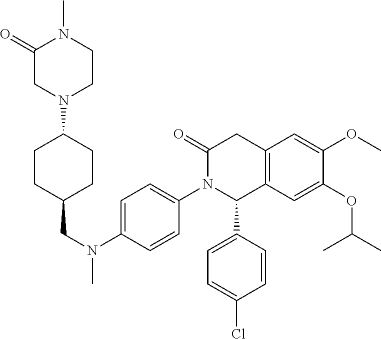 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

(A) or a pharmaceutically acceptable salt thereof.

The compound having the structure of Formula (A) is referred to herein as “Compound (A),” For convenience, the group of the compound having the structure of Formula (A) and possible salts and solvates thereof is collectively referred to to as Compound (A), meaning that reference to Compound (A) will refer to any of the compound or pharmaceutically acceptable salt thereof in the alternative. Compound (A) can be prepared according to WO 2011/076786, which is hereby incorporated by reference in its entirety. Compound (A) was disclosed in WO 2011/076786 as example 106.

In another embodiment, the MDM2 inhibitor is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one inhibits the interaction between MDM2 and p53 while it also inhibits the interaction between MDM4 and p53. Its preparation was described in WO2013/111105, which is hereby incorporated by reference in its entirety. The compound in Example 102 of WO2013/111105 has the structure of Formula (B)

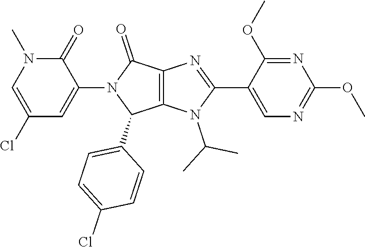 [SMILES: COc1ncc(-c2nc3c(n2C(C)C)[C@H](c2ccc(Cl)cc2)N(c2cc(Cl)cn(C)c2=O)C3=O)c(OC)n1]

(B) and pharmaceutically acceptable salts thereof or a pharmaceutically acceptable non-covalent derivative (including salt, solvate, hydrate, complex, co-crystal) thereof.

The compound having the structure of Formula (B) is referred to herein as “Compound (B).” For convenience, the group of the compound having the structure of Formula (B) and possible salts, solvates, hydrates, complexes, co-crystals are collectively referred to to as Compound (B), meaning that reference to Compound (B) will refer to any of the compound or pharmaceutically acceptable salt or solvate thereof in the alternative.

Compounds (A) and (B) can be generally administered in unit dosage of about 1-5000 mg of active ingredient(s) for a subject of about 50-70 kg, or about 1 mg-3 g or about 1-250 mg or about 1-150 mg or about 0.5-100 mg, or about 1-50 mg of active ingredient. The unit dosage may be administered once or repeatedly during the same day, or during the week. More specifically, daily dose of between 100 mg and 1500 mg, particularly between 300 mg and 1000 mg may be suitable for Compound (A). For Compound (B), doses between 10 mg and 1000 mg may be suitable. Daily doses of the compounds may or may not require drug holidays. For example, the dosing regimen may include 3 weeks on the drug and 1 week off. Also, the dosing regimen may include continuous dosing as disclosed in WO/2015/198266. All dosing regimen disclosed in WO/2015/198266 is hereby incorporated by reference. The combination partners may not be administered according to the same dosing regimen. The compounds (A) or (B) can be used every 3 weeks or every 4 weeks. Particularly compound (B) can be used every 3 weeks. It can also be administered to a patient every 4 weeks. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

As used herein, “ruxolitinib” is the JAK1/JAK2 inhibitor (R)-3-(4-(7H-pyrrolo[2,3-d]pyrimidin-4-yl)-1H-pyrazol-1-yl)-3-cyclopentylpropanenitrile, also named 3(R)-Cyclopentyl-3-[4-(7H-pyrrolo[2,3-d]pyrimidin-4-yl)-1H-pyrazol-1-yl]propanenitrile, of formula:

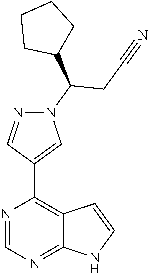 [SMILES: N#CC[C@H](C1CCCC1)n1cc(-c2ncnc3[nH]ccc23)cn1]

which can be prepared, for example, as described in WO2007/070514, which is incorporated herein by reference. As used herein, “ruxolitinib” refers to the free form, and any reference to “a pharmaceutically acceptable salt thereof” refers to “a pharmaceutically acceptable acid addition salt thereof”, in particular ruxolitinib phosphate, which can be prepared, for example, as described in WO2008/157208, which is incorporated herein by reference. Ruxolitinib is approved for the treatment of intermediate to high-risk myelofibrosis under the tradename Jakafi®/Jakavi®.

Ruxolitinib, or pharmaceutically acceptable salt thereof, in particular ruxolitinib phosphate, can be in a unit dosage form (e.g. tablet), which is administered orally.

In one embodiment, “ruxolitinib” is also intended to represent isotopically labeled forms. Isotopically labeled compounds have structures depicted by the formula above except that one or more atoms are replaced by an atom having a selected atomic mass or mass number. Isotopes that can be incorporated into ruxolitinib, for example, isotopes of hydrogen, namely the compound of formula:

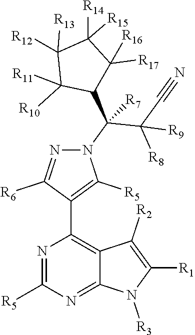

wherein each R₁, R₂, R₃, R₄, R₅, R₆, R₇, R₈, R₉, R₁₀, R₁₁, R₁₂, R₁₃, R₁₄, R₁₅, R₁₆ and R₁₇ is independently selected from H or deuterium; provided that there is at least one deuterium present in the compound. In other embodiments there are multiple deuterium atoms present in the compound. Suitable compounds are disclosed in U.S. Pat. No. 9,249,149 B2, which is hereby incorporated in its entirety.

In one preferred embodiment, a deuterated ruxolitinib is selected from the group consisting of

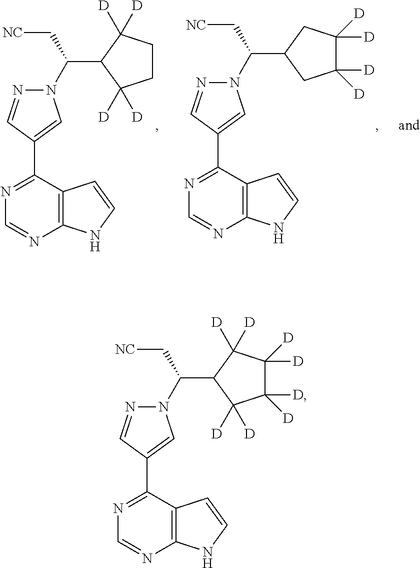

or a pharmaceutically acceptable salt of any of the foregoing.

In a preferred embodiment, a deuterated ruxolitinib is

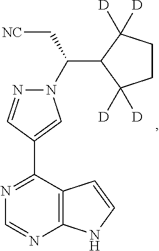 [SMILES: [2H]C1([2H])CCC([2H])([2H])C1[C@@H](CC#N)n1cc(-c2ncnc3[nH]ccc23)cn1]

or a pharmaceutically acceptable salt thereof.

As used herein, “itacitinib” refers to the JAK1/JAK2 inhibitor 2-(3-(4-(7H-pyrrolo(2,3-d)pyrimidin-4-yl)-1H-pyrazol-1-yl)-1-(1-(3-fluoro-2-(trifluoromethyl)isonicotinoyl)piperidin-4-yl)azetidin-3-yl)acetonitrile, also named 2-[1-[1-[3-fluoro-2-(trifluoromethyl)pyridine-4-carbonyl]piperidin-4-yl]-3-[4-(7H-pyrrolo[2,3-d]pyrimidin-4-yl]pyrazol-1-yl]azetidin-3-yl]acetonitrile of formula

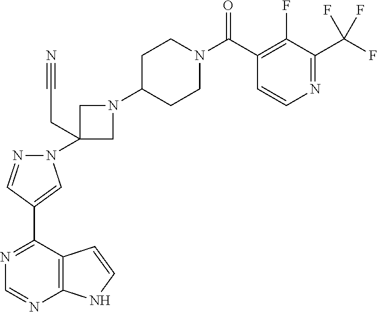 [SMILES: N#CCC1(n2cc(-c3ncnc4[nH]ccc34)cn2)CN(C2CCN(C(=O)c3ccnc(C(F)(F)F)c3F)CC2)C1]

which can be prepared, for example, as described in WO2011/112662, which is incorporated herein by reference. As used herein, “itacitinib” refers to the free form, and any reference to “a pharmaceutically acceptable salt thereof” refers to “a pharmaceutically acceptable acid addition salt thereof”, in particular itacitinib adipate.

### Treatment of Myelofibrosis

In one aspect the present invention provides an MDM2 inhibitor (e.g., siremadlin) or a pharmaceutical acceptable salt thereof, alone or in combination with a JAK inhibitor, (e.g., ruxolitinib) or a pharmaceutical acceptable salt thereof, for use in the treatment of Philadelphia-chromosome negative myeloproliferative neoplasms.

In one further aspect the present invention provides an MDM2 inhibitor (e.g., siremadlin) or a pharmaceutical acceptable salt thereof, for use in the treatment of myelofibrosis (MF) in a patient. Alternatively, in one aspect the present invention provides an MDM2 inhibitor (e.g., siremadlin) or a pharmaceutical acceptable salt thereof, for use in the manufacture of a medicament for the treatment of myelofibrosis (MF) in a patient. Alternatively, in one aspect the present invention provides a method of treating myelofibrosis (MF) in a patient comprising the step of administering therapeutically effective amount of an MDM2 inhibitor (e.g., siremadlin) or a pharmaceutical acceptable salt thereof, to said patient.

Myelofibrosis comprises primary myelofibrosis (PMF), post-essential thrombocythemia myelofibrosis (PET-MF) and post-polycythemia vera myelofibrosis (PPV-MF). Suitably, myelofibrosis is PMF.

The term “primary myelofibrosis” (PMF), as used herein, is defined with reference to “The 2016 revision to the World Health Organization (WHO) classification of myeloid neoplasms and acute leukemia”, as published in Blood, 2016, 127:2391-2405. Primary myelofibrosis encompasses prefibrotic/early primary myelofibrosis (prePMF) and overt primary myelofibrosis (overt PMF). Diagnosis of prePMF requires meeting the following 3 major criteria, and at least 1 minor criterion according to the 2016 WHO classification for prePMF in table 1:

TABLE 1
Criteria for diagnosis of prePMF

Major criteria (prePMF)
1. Megakaryocytic proliferation and atypia, without reticulin
fibrosis >grade 1, accompanied by increased age-adjusted BM
cellularity, granulocytic proliferation, and often decreased
erythropoiesis
2. Not meeting the WHO criteria for BCR-ABL1⁺CML, PV, ET,
myelodysplastic syndromes, or other myeloid neoplasms
3. Presence of JAK2, CALR, or MPL mutation or in the absence
of these mutations, presence of another clonal marker (e.g.,
ASXL1, EZH2, TET2, IDH1/IDH2, SRSF2, SF3B1) are of help in
determining the clonal nature of the disease or absence of
minor reactive bone marrow (BM) reticulin fibrosis (Minor
(grade 1) reticulin fibrosis secondary to infection, autoimmune
disorder or other chronic inflammatory conditions, hairy cell
leukemia or other lymphoid neoplasm, metastatic malignancy, or
toxic (chronic) myelopathies)
Minor criteria (prePMF)
Presence of at least 1 of the following, confirmed in 2
consecutive determinations:
a. Anemia not attributed to a comorbid condition
b. Leukocytosis ≥11*10⁹/L
c. Palpable splenomegaly
d. LDH increased to above upper normal limit of institutional
reference range

Diagnosis of overt PMF requires meeting the following 3 major criteria, and at least 1 minor criterion according to the 2016 WHO classification for overt PMF in table 2:

TABLE 2
Criteria for diagnosis of overt PMF

Major criteria (overt PMF)
1. Presence of megakaryocytic proliferation and atypia,
accompanied by either reticulin and/or collagen fibrosis
grades 2 or 3
2. Not meeting WHO criteria for ET, PV, BCR-ABL1⁺CML,
myelodysplastic syndromes, or other myeloid neoplasms
3. Presence of JAK2, CALR, or MPL mutation or in the absence of
these mutations, presence of another clonal marker (e.g., ASXL1,
EZH2, TET2, IDH1/IDH2, SRSF2, SF3B1) or absence of reactive
myelofibrosis (BM fibrosis secondary to infection, autoimmune
disorder, or other chronic inflammatory conditions, hairy cell
leukemia or other lymphoid neoplasm, metastatic malignancy, or
toxic (chronic) myelopathies)
Minor criteria (overt PMF)
Presence of at least 1 of the following,
confirmed in 2 consecutive determinations:
a. Anemia not attributed to a comorbid condition
b. Leukocytosis ≥11*10⁹/L
c. Palpable splenomegaly
d. LDH increased to above upper normal limit
of institutional reference range
e. Leukoerythroblastosis

The term “bone marrow fibrosis”, as used herein, refers to bone marrow fibrosis graded according to the 2005 European consensus grading system (Thiele et. al., Haematologica, 2005, 90(8), 1128-1132, in particular as defined in Table 3 and FIG. 1 of page 1130 therein), such as:
“fibrosis grade 0”: scattered linear reticulin with no intersections (cross-overs) corresponding to normal bone marrow;
“fibrosis grade 1”: loose network of reticulin with many intersections, especially in perivascular areas;
“fibrosis grade 2”: diffuse and dense increase in reticulin with extensive intersections, occasionally with only focal bundles of collagen and/or focal osteosclerosis;
“fibrosis grade 3”: diffuse and dense increase in reticulin with extensive intersections with coarse bundles of collagen, often associated with significant osteosclerosis;
wherein the grading (i.e. grading of fiber density and quality) is made on the basis of bone marrow biopsy specimen assessment.

The term “essential thrombocythemia” (ET), as used herein, is defined with reference to “The 2016 revision to the World Health Organization (WHO) classification of myeloid neoplasms and acute leukemia”, as published in Blood, 2016, 127:2391-2405. The term “post-essential thrombocythemia myelofibrosis” (PET-MF), as used herein, refers to MF secondary to ET (i.e. MF arising as a progression of ET), wherein ET is as defined herein above. According to the IWG-MRT criteria (Barosi G et al, Leukemia (2008) 22, 437-438), criteria for diagnosing post-essential thrombocythemia myelofibrosis are:

TABLE 3
Criteria for diagnosis of post-essential
thrombocythemia myelofibrosis

Required criteria:
1. Documentation of a previous diagnosis of essential thrombocythemia
as defined by the WHO criteria
2. Bone marrow fibrosis grade 2-3
Additional criteria (two are required):
1. Anemia and a ≥2 mg/ml decrease from baseline hemoglobin level
2. A leukoerythroblastic peripheral blood picture
3. Increasing splenomegaly defined as either an increase in palpable
splenomegaly of ≥5 cm (distance of the tip of the spleen from the
left costal margin) or the appearance of a newly palpable splenomegaly
4. Increased lactate dehydrogenase (LDH) (above reference level)
5. Development of ≥1 of three constitutional symptoms: >10% weight
loss in 6 months, night sweats, unexplained fever (>37.5° C.)

The term “polycythemia vera” (PV), as used herein, is defined with reference to “The 2016 revision to the World Health Organization (WHO) classification of myeloid neoplasms and acute leukemia”, as published in Blood, 2016, 127:2391-2405. The term “post-polycythemia myelofibrosis” (PPV-MF), as used herein, refers to MF secondary to PV (i.e. MF arising as a progression of PV). According to the IWG-MRT criteria (Barosi G et al, Leukemia (2008) 22, 437-438), criteria for diagnosing post-polycythemia myelofibrosis are:

TABLE 4
Criteria for diagnosis of post-polycythemia myelofibrosis

Required criteria:
1. Documentation of a previous diagnosis of polycythemia
vera as defined by the WHO criteria
2. Bone marrow fibrosis grade 2-3 (on 0-3 scale)
Additional criteria (two are required):
1. Anemia or sustained loss of requirement of either
phlebotomy (in the absence of cytoreductive therapy)
or cytoreductive treatment for erythrocytes is
2. A leukoerythroblastic peripheral blood picture
3. Increasing splenomegaly defined as either an increase
in palpable splenomegaly of ≥5 cm (distance of the tip
of the spleen from the left costal margin) or the appearance
of a newly palpable splenomegaly
4. Development of ≥1 of three constitutional symptoms: >10%
weight loss in 6 months, night sweats, unexplained fever (>37.5° C.)

As used herein, the following response criteria as defined by the International Working Group-Myeloproliferative Neoplasms Research and Treatment (IWG-MRT) and the European Leukemia Net (ELN) response criteria for MF (Tefferi et al, Blood 2013 122:1395-1398, which is incorporated by reference in its entirety) are used herein:

TABLE 5
International Working Group-Myeloproliferative Neoplasms
Research and Treatment (IWG-MRT) and the European Leukemia
Net (ELN) response criteria for myelofibrosis

| | Required criteria (for all response |
|---|---|
| Response | categories, benefit must last for ≥12 |
| categories | weeks to qualify as a response) |
| Complete | Bone marrow: *Age-adjusted normocellularity; <5% |
| response | blasts; ≤grade 1 MF^{†} and |
| (CR) | Peripheral blood: Hemoglobin ≥100 g/L and <UNL; |
| | neutrophil count ≥1 × 10⁹/L and <UNL; |
| | Platelet count ≥100 × 10⁹/L and <UNL; <2% |
| | immature myeloid cells^{‡} and |
| | Clinical: Resolution of disease symptoms; spleen |
| | and liver not palpable; no evidence of EMH |
| Partial | Peripheral blood: Hemoglobin ≥100 g/L and <UNL; |
| response | neutrophil count ≥1 × 10⁹/L and <UNL; platelet |
| (PR) | count ≥100 × 10⁹/L and <UNL; <2% immature |
| | myeloid cells^{‡} and |
| | Clinical: Resolution of disease symptoms; spleen and |
| | liver not palpable; no evidence of EMH or |
| | Bone marrow: *Age-adjusted normocellularity; <5% |
| | blasts; ≤grade 1 MF^{†}, and peripheral blood: |
| | Hemoglobin ≥85 but <100 g/L and <UNL; neutrophil |
| | count ≥1 × 10⁹/L and <UNL; platelet count ≥50, |
| | but <100 × 10⁹/L and <UNL; <2% immature myeloid |
| | cells^{‡} and |
| | Clinical: Resolution of disease symptoms; spleen |
| | and liver not palpable; no evidence of EMH |
| Clinical | The achievement of anemia, spleen or symptoms response |
| improvement | without progressive disease or increase in severity of |
| (CI) | anemia, thrombocytopenia, or neutropenia^{§} |
| Anemia | Transfusion-independent patients: a ≥20 g/L increase |
| response | in hemoglobin level^{∥} |
| | Transfusion-dependent patients: becoming transfusion- |
| | independent^{¶} |
| Spleen | A baseline splenomegaly that is palpable at 5-10 cm, |
| response^{#} | below the LCM, becomes not palpable** or |
| | A baseline splenomegaly that is palpable at >10 cm, |
| | below the LCM, decreases by ≥50%** |
| | A baseline splenomegaly that is palpable at <5 cm, |
| | below the LCM, is not eligible for spleen response |
| | A spleen response requires confirmation by MRI or |
| | computed tomography showing ≥35% spleen volume |
| | reduction |
| Symptoms | A ≥50% reduction in the MPN-SAF TSS^{††} |
| response | |

EMH, extramedullary hematopoiesis (no evidence of EMH implies the absence of pathology- or imaging study-proven nonhepatosplenic EMH); LCM, left costal margin; UNL, upper normal limit.
*Baseline and posttreatment bone marrow slides are to be interpreted at one sitting by a central review process.
^{†}Grading of MF is according to the European classification: Thiele et al. European consensus on grading bone marrow fibrosis and assessment of cellularity. Haematologica. 2005; 90: 1128.
^{‡}Immature myeloid cells constitute blasts + promyelocytes + myelocytes + metamyelocytes + nucleated red blood cells. In splenectomized patients, <5% immature myeloid cells is allowed.
^{§}Increase in severity of anemia constitutes the occurrence of new transfusion dependency or a ≥20 g/L decrease in hemoglobin level from pretreatment baseline that lasts for at least 12 weeks. Increase in severity of thrombocytopenia or neutropenia is defined as a 2-grade decline, from pretreatment baseline, in platelet count or absolute neutrophil count, according to the Common Terminology Criteria for Adverse Events (CTCAE) version 4.0. In addition, assignment to CI requires a minimum platelet count of ≥25 000 × 10(9)/L and absolute neutrophil count of ≥0.5 × 10(9)/L.
^{∥}Applicable only to patients with baseline hemoglobin of <100 g/L. In patients not meeting the strict criteria for transfusion dependency at the time of treatment initiation, but have received transfusions within the previous month, the pre-transfusion hemoglobin level should be used as the baseline.
^{¶}Transfusion dependency is defined as transfusions of at least 6 units of packed red blood cells (PRBC), in the 12 weeks prior to start of treatment initiation, for a hemoglobin level of <85 g/L, in the absence of bleeding or treatment-induced anemia. In addition, the most recent transfusion episode must have occurred in the 28 days prior to start of treatment initiation. Response in transfusion-dependent patients requires absence of any PRBC transfusions during any consecutive “rolling” 12-week interval during the treatment phase, capped by a hemoglobin level of ≥85 g/L.
^{#}In splenectomized patients, palpable hepatomegaly is substituted with the same measurement strategy.
**Spleen or liver responses must be confirmed by imaging studies where a ≥35% reduction in spleen volume, as assessed by MRI or CT, is required. Furthermore, a ≥35% volume reduction in the spleen or liver, by MRI or CT, constitutes a response regardless of what is reported with physical examination.
^{††}Symptoms are evaluated by the MPN-SAF TSS. The MPN-SAF TSS is assessed by the patients themselves and this includes fatigue, concentration, early satiety, inactivity, night sweats, itching, bone pain, abdominal discomfort, weight loss, and fevers. Scoring is from 0 (absent/as good as it can be) to 10 (worst imaginable/as bad as it can be) for each item. The MPN-SAF TSS is the summation of all the individual scores (0-100 scale). Symptoms response requires ≥50% reduction in the MPN-SAF TSS.

In one embodiment the present invention provides an MDM2 inhibitor, suitably siremadlin, alone or in combination with a JAK inhibitor, suitably ruxolitinib or a pharmaceutically acceptable salt thereof, for use in the treatment of myelofibrosis, especially primary MF, wherein the patient achieves complete response to the treatment according to the criteria in Table 5.

In one embodiment the present invention provides an MDM2 inhibitor, suitably siremadlin, alone or in combination with a JAK inhibitor, suitably ruxolitinib or a pharmaceutically acceptable salt thereof, for use in the treatment of myelofibrosis, especially primary MF, wherein the patient achieves partial response to the treatment according to the criteria in Table 5.

Among patients, myelofibrosis frequently causes shortened survival due to disease transformation to acute leukemia, progression without acute transformation, cardiovascular complications or thrombosis, infection or portal hypertension. It is one of the aims of the present invention to improve the median survival of myelofibrosis patients.

As used herein, the term “median survival time” refers to the time of diagnosis or from the time of initiation of treatment according to the present invention that half of the patients in a group of patients diagnosed with the disease are still alive compared to patients receiving best available treatment or compared to patients receiving placebo and wherein patients belong to the same risk group of myelofibrosis, for example as described by Gangat et al (J Clin Oncol. 2011 Feb. 1; 29(4):392-397), which is hereby incorporated by reference in its entirety.

Accordingly, in one embodiment the present invention provides an MDM2 inhibitor, suitably siremadlin, alone or in combination with a JAK inhibitor, suitably ruxolitinib or a pharmaceutically acceptable salt thereof, for use in the treatment of myelofibrosis, especially primary MF, wherein median survival time is increased by at least 3 months in the group of high risk MF patients or by at least six months, preferably by at least 12 months in the group of medium risk MF patients.

As used herein, the term “subject” refers to a human being.

The term “treat”, “treating”, “treatment” or “therapy”, as used herein, means obtaining beneficial or desired results, for example, clinical results. Beneficial or desired results can include, but are not limited to, alleviation of one or more symptoms, as defined herein. One aspect of the treatment is, for example, that said treatment should have a minimal adverse effect on the patient, e.g. the agent used should have a high level of safety, for example without producing the side effects of a previously known therapy. The term “alleviation”, for example in reference to a symptom of a condition, as used herein, refers to reducing at least one of the frequency and amplitude of a symptom of a condition in a patient.

As used herein, the term “newly diagnosed” refers to diagnosis of the disorder, e.g. myelofibrosis and said patient has not received any treatment. In one embodiment the present invention provides an MDM2 inhibitor (e.g., siremadlin) or a pharmaceutical acceptable salt thereof, alone or in combination with a JAK inhibitor, suitably ruxolitinib or a pharmaceutically acceptable salt thereof, for use in the treatment of a newly diagnosed myelofibrosis patient

The term “triple-negative myelofibrosis patient”, as used herein, refers to a patient who lacks JAK2, CALR and MPL mutations. In one embodiment the present invention provides an MDM2 inhibitor (e.g., siremadlin) or a pharmaceutical acceptable salt thereof, alone or in combination with a JAK inhibitor, suitably ruxolitinib or a pharmaceutically acceptable salt thereof, for use in the treatment of triple-negative myelofibrosis patient.

The term “best available therapy”, as used herein, refers to any commercially available agent approved prior to March 2018 for the treatment of PMF, PET-MF or PPV-MF, as monotherapy, or in combination. Exemplary agents include, but are not limited to ruxolitinib or a pharmaceutically acceptable salt thereof, antineoplastic agents (e.g., hydroxyurea, anagrelide), glucocorticoids prednisone/prednisolone, methylprednisolone), antianemia preparations (e.g., epoetin-alpha), immunomodulatory agents (e.g., thalidomide, lenalidomide), purine analogs (e.g., mercaptopurine, thioguanine), antigonadotropins (e.g., danazol), interferons (e.g., PEG-interferon-alpha 2a, interferon-alpha), nitrogen mustard analogs (e.g. melphalan), pyrimidine analogs (e.g., cytarabine).

The term “splenomegaly”, as used herein, refers to a palpably enlarged spleen (e.g. a spleen is palpable at 5 cm below the left coastal margin) or to an enlarged spleen as detected by an imaging test (e.g. a computed tomography (CT) scan, MRI, X-rays or ultrasound), wherein the term “enlarged spleen” refers to a spleen greater in size than normal (e.g., median normal spleen volume of 200 cm³).

The term “treatment of splenomegaly”, as used herein, refers to “improvement of splenomegaly”, which means a decrease in splenomegaly, for example a reduction in spleen volume, as defined by the International Working Group-Myeloproliferative Neoplasms Research and Treatment (IWG-MRT) and the European Leukemia Net (ELN) response criteria for MF in Table 5. In one embodiment, the invention may provide the use of an MDM2 inhibitor (e.g., siremadlin) or a pharmaceutical acceptable salt thereof, alone or in combination with ruxolitinib or a pharmaceutically acceptable salt thereof for treatment of myelofibrosis, particularly for the treatment of splenomegaly associated with myelofibrosis, resulting in, for example, ≥20%, ≥25%, ≥30% or ≥35% reduction in spleen volume as measured by magnetic resonance imaging (MRI) or computed tomography (CT) from pre-treatment baseline to, for example, week 24 or week 48.

The term “hepatomegaly”, as used herein, refers to a palpably enlarged liver or to an enlarged liver as detected by an imaging test (e.g. a computed tomography (CT) scan), wherein the term “enlarged liver” refers to a liver greater in size than normal (e.g., median normal liver volume of approximately 1500 cm³).

The term “treatment of hepatomegaly”, as used herein, refers to “improvement of hepatomegaly”, which means a decrease in hepatomegaly, for example a reduction in hepatomegaly, as defined according to the International Working Group-Myeloproliferative Neoplasms Research and Treatment (IWG-MRT) and the European Leukemia Net (ELN) response criteria for MF in the preceding table. Accordingly, in one embodiment the present invention provides the use of an MDM2 inhibitor (e.g., siremadlin) or a pharmaceutical acceptable salt thereof, alone or in combination with ruxolitinib or a pharmaceutically acceptable salt thereof for treatment of myelofibrosis, particularly for the treatment of hepatomegaly associated with myelofibrosis, resulting in, for example, ≥20%, ≥25%, ≥30% or ≥35% reduction in liver volume as measured by magnetic resonance imaging (MRI) or computed tomography (CT) from pre-treatment baseline to, for example, week 24 or week 48.

The term “thrombocytopenia”, as used herein, refers to a platelet count, in blood specimen laboratory test, lower than normal. The term “severity of thrombocytopenia”, as used herein, refers, for example, to specific grade 1-4 of thrombocytopenia according to CTCAE (version 4.03).

The term “treatment of thrombocytopenia”, as used herein, refers to “stabilizing thrombocytopenia” or “improving thrombocytopenia”, in comparison to the pre-treatment situation or in comparison to best available therapy or to placebo control. The term “stabilizing thrombocytopenia” refers, for example, to prevent an increase in the severity of thrombocytopenia, namely the platelet count remains stable. The term “improving thrombocytopenia” refers to alleviation of the severity of thrombocytopenia, namely increasing blood platelet count. In one embodiment, the invention provides an MDM2 inhibitor (e.g., siremadlin) or a pharmaceutical acceptable salt thereof, alone or in combination with ruxolitinib or a pharmaceutically acceptable salt thereof, for use in the treatment of myelofibrosis, particularly for the treatment of thrombocytopenia associated with myelofibrosis, resulting in stabilizing thrombocytopenia or improving thrombocytopenia from pre-treatment baseline to, for example, week 24 or week 48 of treatment.

The term “neutropenia”, as used herein, refers to an absolute neutrophil count (ANC), in blood specimen laboratory test, lower than normal value. The term “severity of neutropenia”, as used herein, refers, for example, to specific grade 1-4 of neutropenia according to CTCAE (version 4.03).

The term “treatment of neutropenia”, as used herein, refers to “stabilizing neutropenia” or “improving neutropenia”, for example, in comparison to the pre-treatment situation or in comparison to best available therapy or to placebo control. The term “stabilizing neutropenia” refers, for example, to prevent an increase in the severity of neutropenia. The term “improving neutropenia” refers, for example, to a decrease in the severity of neutropenia. In one embodiment, the invention provides an MDM2 inhibitor (e.g., siremadlin) or a pharmaceutical acceptable salt thereof, with ruxolitinib or a pharmaceutically acceptable salt thereof, for use in the treatment of myelofibrosis, particularly for the treatment of neutropenia associated with myelofibrosis, resulting in stabilizing neutropenia or improving neutropenia from pre-treatment baseline to, for example, week 24 or week 48 of treatment.

The term “anemia”, as used herein, refers to hemoglobin level, in blood specimen laboratory test, of less than 13.5 gram/100 ml in men and hemoglobin level of less than 12.0 gram/100 ml in women. The term “severity of anemia”, as used herein, refers, for example, to specific grade 1-4 of anemia according to CTCAE (version 4.03)].

The term “treatment of anemia”, as used herein, refers to “stabilizing anemia” or “improving anemia”, for example, in comparison to the pre-treatment situation or in comparison to best available therapy or to placebo control. The term “stabilizing anemia” refers, for example, to prevent an increase in the severity of anemia (e.g. preventing that a “transfusion-independent” patient becomes a “transfusion-dependent” patient or preventing anemia grade 2 becomes anemia grade 3). The term “improving anemia” refers to a decrease in the severity of anemia or an improvement in hemoglobin level. In one embodiment, the invention may provide the use of an MDM2 inhibitor (e.g., siremadlin) or a pharmaceutical acceptable salt thereof, alone or in combination with ruxolitinib or a pharmaceutically acceptable salt thereof, for treatment of myelofibrosis, particularly for the treatment of anemia associated with myelofibrosis, resulting in stabilizing anemia or improving anemia from pre-treatment baseline to, for example, week 24 or week 48 of treatment.

The term “treatment of bone marrow fibrosis associated with MF”, as used herein, means “stabilizing bone marrow fibrosis” or “improving bone marrow fibrosis”, for example, in comparison to the pre-treatment situation or in comparison to best available therapy or to placebo control. The term “stabilizing bone marrow fibrosis” refers, for example, to prevent increase in severity of bone marrow fibrosis. The term “improving bone marrow fibrosis” refers to a decrease in severity of bone marrow fibrosis, for example, from pre-treatment baseline, according to the 2005 European consensus grading system. In one embodiment, the invention may provide the use of an MDM2 inhibitor (e.g., siremadlin) or a pharmaceutical acceptable salt thereof, alone or in combination with ruxolitinib or a pharmaceutically acceptable salt thereof, for treatment of myelofibrosis, particularly for the treatment of bone marrow fibrosis associated with MF, resulting in stabilizing bone marrow fibrosis or improving bone marrow fibrosis from pre-treatment baseline to, for example, week 24 or week 48 of treatment.

The term “constitutional symptoms associated with myelofibrosis”, as used herein, refers to common debilitating chronic myelofibrosis symptoms, such as fever, pruritus (i.e. itching), abdominal pain/discomfort, weight loss, fatigue, inactivity, early satiety, night sweats or bone pain; for example, as described by Mughal et al (Int J Gen Med. 2014 Jan. 29; 7:89-101).

The term “treatment of constitutional symptoms associated with myelofibrosis”, as used herein, refers to “improvement of constitutional symptoms associated with myelofibrosis”, for example, in comparison to the pre-treatment situation or in comparison to best available therapy or to placebo control, for example, a reduction in total symptom score as measured by the modified myelofibrosis symptom assessment form version 2.0 diary (modified MFSAF v2.0) (Cancer 2011; 117:4869-77; N Engl J Med 2012; 366:799-807, the entire contents of which are incorporated herein by reference). In one embodiment, the invention may provide the use of an MDM2 inhibitor (e.g., siremadlin) or a pharmaceutical acceptable salt thereof, alone or in combination with ruxolitinib or a pharmaceutically acceptable salt thereof, for treatment of myelofibrosis, particularly for the treatment of constitutional symptoms associated with myelofibrosis, resulting in improvement of constitutional symptoms associated with myelofibrosis from pre-treatment baseline to, for example, week 24 or week 48 of treatment.

In another embodiment of any use of the invention, one or more of the constitutional symptoms associated with MF are alleviated (e.g. by eliminating or by reducing intensity, duration or frequency). In one embodiment, the reduction of constitutional symptoms is at least ≥20%, at least ≥30%, at least ≥40% or at least ≥50% as assessed by the modified MFSAF v2.0 from pre-treatment baseline to, for example, week 24 or week 48.

In one embodiment of any use of the invention, the MDM2 inhibitor, suitably siremadlin, is administered subsequently or prior to splenectomy or radiotherapy, such as splenic irradiation.

### Combination Therapy

In one aspect the present invention provides an MDM2 inhibitor, suitably siremadlin, for use in the treatment of MF, wherein said MDM2 inhibitor is administered in combination with at least one further active agent.

In one embodiment the at least one agent is an inhibitor of a non-receptor tyrosine kinases, the Janus kinases (JAK). A considerable number of cytokine and growth factor receptors utilize non-receptor tyrosine kinases, the Janus kinases (JAK), to transmit extracellular ligand binding into an intracellular response. For example, erythropoietin, thrombopoietin and granulocyte monocyte colony stimulating factor are all known to signal through receptors that utilize JAK2. JAK activate a number of downstream pathways implicated in proliferation and survival, including the STATs (signal transducers and activators of transcription), a family of important latent transcription factors.

Accordingly, the present invention relates to the combination use of an MDM2 inhibitor (e.g., siremadlin) or a pharmaceutical acceptable salt thereof, with at least one JAK inhibitor, suitably ruxolitinib or a pharmaceutically acceptable salt thereof.

In one embodiment the at least one further active agent is a JAK1/JAK2 inhibitor, suitably ruxolitinib or a pharmaceutically acceptable salt thereof or momelotinib or a pharmaceutically acceptable salt thereof, more suitably ruxolitinib or a pharmaceutically acceptable salt, more suitably ruxolitinib phosphate.

Ruxolitinib represents a novel, potent, and selective inhibitor of JAK1 and JAK2.

Ruxolitinib potently inhibits JAK1 and JAK2 [half maximal inhibitory concentration (IC50) 0.4 to 1.7 nM], yet it does not significantly inhibit (<30% inhibition) a broad panel of 26 kinases when tested at 200 nM (approximately 100× the average IC50 value for JAK enzyme inhibition) and does not inhibit JAK3 at clinically relevant concentrations.

In one embodiment the at least one further active agent is a JAK2/FLT3 inhibitor, suitably pacritinib or a pharmaceutically acceptable salt thereof or fedratinib or a pharmaceutically acceptable salt thereof.

In one embodiment the at least one further active agent is a JAK2^{V617F} inhibitor, suitably gandotinib or a pharmaceutically acceptable salt thereof.

In one embodiment the at least one further active agent is a JAK2 inhibitor, suitably BMS-911543 or a pharmaceutically acceptable salt thereof.

In one embodiment the at least one further active agent is a JAK1 inhibitor, suitably itacitinib or a pharmaceutically acceptable salt thereof, in particular itacitinib adipate.

In one embodiment the at least one further active agent is a JAK2/Src inhibitor, suitably NS-018 or a pharmaceutically acceptable salt thereof.

In one aspect the present invention provides a pharmaceutical combination, separate, comprising, consisting essentially of or consisting of siremadlin or a pharmaceutical acceptable salt thereof, and b) a JAK1/2 inhibitor, suitably ruxolitinib or a pharmaceutically acceptable salt thereof. Suitably the pharmaceutical combination is for use in the treatment of myelofibrosis.

In one aspect the present invention provides siremadlin or a pharmaceutical acceptable salt thereof for use in the treatment of myelofibrosis, wherein siremadlin or a pharmaceutical acceptable salt thereof, is administered in combination with ruxolitinib or a pharmaceutically acceptable salt thereof, and wherein siremadlin or a pharmaceutical acceptable salt thereof and ruxolitinib or a pharmaceutically acceptable salt thereof, are administered in jointly therapeutically effective amounts.

In one aspect the present invention provides ruxolitinib or a pharmaceutically acceptable salt thereof, for use in the treatment of myelofibrosis, wherein ruxolitinib or a pharmaceutically acceptable salt thereof, is administered in combination with siremadlin or a pharmaceutical acceptable salt thereof, and wherein ruxolitinib or a pharmaceutically acceptable salt thereof, and siremadlin or a pharmaceutical acceptable salt thereof, are administered in jointly therapeutically effective amounts.

The term “combination” or “pharmaceutical combination” used herein, refers to a non-fixed combination where an active agent and at least one further active agent may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “non-fixed combination” means that the active ingredients, e.g. one active agent and at least one further active agent, are both administered to a patient as separate entities either simultaneously or sequentially with no specific time limits, wherein such administration provides therapeutically effective levels of the two compounds in the body of the patient. In particular, reference to siremadlin or a pharmaceutical acceptable salt thereof in combination with ruxolitinib or a pharmaceutically acceptable salt thereof as used herein (e.g. in any of the embodiments or in any of the claims herein), refers to a “non-fixed combination”; and reference to ruxolitinib or a pharmaceutically acceptable salt thereof as used herein (e.g. in any of the embodiments or in any of the claims herein), in combination with at least one further active agent (siremadlin being excluded) refers to either a fixed combination in one unit dosage form (e.g., capsule, tablet, caplets or particulates), a non-fixed combination, or a kit-of-parts for the combined administration wherein ruxolitinib or a pharmaceutically acceptable salt thereof and one or more combination partner (e.g. another drug as specified herein, also referred to as further “pharmaceutical active ingredient”, “therapeutic agent” or “co-agent”) may be administered independently at the same time or separately within time intervals.

The term “therapeutically effective amount” refers to an amount of a drug or a therapeutic agent that will elicit the desired biological and/or medical response of a tissue, system or an animal (including man) that is being sought by a researcher or clinician.

### Administration and Treatment Regimen

Compounds (A) and (B) can be generally administered in unit dosage of about 1-5000 mg of active ingredient(s) for a subject of about 50-70 kg, or about 1 mg-3 g or about 1-250 mg or about 1-150 mg or about 0.5-100 mg, or about 1-50 mg of active ingredient. The unit dosage may be administered once or repeatedly during the same day, or during the week. More specifically, daily dose of between 100 mg and 1500 mg, particularly between 300 mg and 1000 mg may be suitable for Compound (A). For Compound (B), doses between 10 mg and 1000 mg may be suitable. Daily doses of the compounds may or may not require drug holidays. For example, the dosing regimen may include 3 weeks on the drug and 1 week off. The combination partners may not be administered according to the same dosing regimen. The compounds (A) or (B) can be used every 3 weeks or every 4 weeks. Particularly compound (B) can be used every 3 weeks. It can also be administered to a patient every 4 weeks. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

In one embodiment the present invention provides an MDM2 inhibitor such as siremadlin or a pharmaceutical acceptable salt thereof, for use in the treatment of myelofibrosis, wherein said MDM2 inhibitor, is administered in combination with ruxolitinib, or a pharmaceutically acceptable salt thereof. Suitably ruxolitinib is administered in an amount of from 5 mg twice daily to 25 mg twice daily, such as 5 mg twice daily, 10 mg twice daily, 15 mg twice daily, 20 mg twice daily or 25 mg twice daily, depending on the patient's blood count according to the prescribing information for Jakavi®/Jakafi® and the judgment of the treating physician.

All publications and patent applications mentioned in the specification are indicative of the level of skill of those skilled in the art to which the presently disclosed inventive concepts pertain. All publications and patent applications are herein incorporated by reference to the same extent as if each individual publication or patent application was specifically and individually indicated to be incorporated by reference.

### LIST OF ABBREVIATIONS

Ab antibody
AE adverse event
AML Acute myeloid leukemia
ANC Absolute neutrophil count
ASCT allogeneic hematopoietic stem cell transplantation
AUC Area under curve
BID twice a day
BM bone marrow
C1D1 Cycle 1 Day 1 (and sequentially for other cycles and days, eg C1D2, C2D1 etc.)
CT computed tomography

### CTCAE Common Terminology Criteria for Adverse Events

CYP cytochrome P-450
DDI Drug-drug interaction
DLT dose-limiting toxicity

### ECG Electrocardiogram

### EORTC European Organization for Research and Treatment of Cancer

ET essential thrombocythemia
Hb hemoglobin

### HDM2 Human Double Minute-2

### IV Intravenous

### IWG-MRT International Working Group-Myeloproliferative Neoplasms Research and Treatment

JAK Janus kinase
LCM left costal margin
MF myelofibrosis
MPN myeloproliferative neoplasm
MRI magnetic resonance imaging
PD pharmacodynamic(s)
PFS progression free survival
PK pharmacokinetic(s)
PLT platelets
PMF primary myelofibrosis
PRBC packed red blood cells
PV polycythemia vera
QD once a day

### QLQ-C30 Quality of Life Questionnaire-Core 30

QoL quality of life
RBC red blood cell(s)
RP2D recommended phase 2 dose
RR Response rate
SAF symptom assessment form
STAT signal transducer and activator of transcription
TLS Tumor lysis syndrome
TSS total symptom score

### WHO World Health Organization

The Examples below are set forth to aid in the understanding of the inventions but are not intended to, and should not be construed to, limit its scope in any way.

### EXPERIMENTAL

### A Randomized, Open-Label, Phase I/II Open Platform Study Evaluating Safety and Efficacy of Novel Ruxolitinib Combination in Myelofibrosis Patients

Rationale for Dose/Regimen and Duration of Treatment for Siremadlin in Combination with Ruxolitinib

This is the first trial that will evaluate the combination of siremadlin with ruxolitinib.

In terms of overlapping enzymes that may potentiate a PK drug-drug interaction (DDI) between siremadlin and ruxolitinib, siremadlin is predominantly metabolized by CYP3A4, is a substrate of P-gp and BCRP in vitro, and is both a time-dependent inhibitor and a reversible inhibitor of CYP3A4/5 in vitro. Siremadlin is also an inducer of CYP3A4/5 in vitro, with a net effect anticipated rather as an inhibitor than an inducer. Ruxolitinib is predominantly metabolized by CYP3A4, and with little or no capacity to inhibit other major CYP enzymes or transporters.

A PK DDI between ruxolitinib and siremadlin is unlikely or predicted to be low, based on a physiologically based pharmacokinetic (PBPK) model (SimCyp) analysis.

Under the planned combination dosing for siremadlin (10, 20, 30 or 40 mg daily from day 1 to 5, 28-day cycle) and ruxolitinib (between 5 and 25 mg BID), a minimal DDI effect is anticipated with a predicted <1.3-fold transient increase of ruxolitinib exposure (AUC and Cmax), through CYP3A4 inhibition. This transient and limited increase in the exposure of ruxolitinib is unlikely to necessitate a ruxolitinib dose adjustment during the period of co-administration with siremadlin. No change of siremadlin systemic exposure is expected in the presence of ruxolitinib. Nonetheless, full PK assessments of ruxolitinib and siremadlin are implemented in the dose escalation phase, and any need for a dose adjustment of either ruxolitinib or siremadlin would be assessed based on emerging PK data.

Ruxolitinib is associated with partial transient hematoxicity. For siremadlin, the most frequently reported adverse drug reactions in clinic were hematological toxicities including thrombocytopenia, neutropenia and anemia. Thus, when siremadlin and ruxolitinib are administered concurrently the potential may occur for additive hematoxicity and will warrant careful monitoring.

In this study, the selection of the siremadlin dose and regimen is based on the currently available preclinical and clinical safety, efficacy, PK and PK/PD modeling information from the phase I study HDM201X2101, a dose escalation and expansion study of single-agent siremadlin in patients with solid tumors or hematologic malignancies (R/R AML). At the time of data cut-off (15 Jan. 2018), 199 subjects have been treated applying various dosing regimens: 115 subjects with solid and 84 patients with hematological tumors. Safety and efficacy data from this trial in AML subjects was used to determine a recommended Phase 2 dose regimen of 45 mg once a day (QD) from day 1 to day 7 of a 28-day cycle (regimen 2C) which is disclosed in WO/2018/178925 (incorporated herewith by reference), At this dose regimen, Complete Response/Complete Response with incomplete blood count recovery (CR/CRi) were respectively 2/4 subjects out of 24 (25%). Detailed review of the safety profile from hematological tumor patients enrolled in regimen 2C did not reveal any unexpected toxicities.

Adverse events were generally mild to moderate in severity, reversible and manageable, with cytopenias being the most commonly observed adverse events. The only DLT was tumor lysis syndrome (TLS). Therefore, regimen 2C is used in this study as the basis for selection of the starting dose and regimen in combination with ruxolitinib in MF subjects. Moreover, preclinical PK/PD tumor growth inhibition modeling of rat xenograft data, as well as clinical PK/PD modeling of tumor growth and bone marrow blast data from solid and hematological tumors, has shown that reducing the application of siremadlin from 7 to 5 consecutive days per 28-day cycle, maintains equivalent anti-tumor activity (Guerreiro et al 2018, Meille et al 2017).

Siremadlin efficacy appears to be primarily driven by cumulative exposure per cycle and thus is probably independent of the selected treatment regimen selected (Guerreiro et al 2018, Meille Clinical testing of siremadlin, alone or in combination with ruxolitinib, are conducted, for example, according to standard clinical practice (e.g. placebo control study, for example in analogy to COMFORT-1 trial) in patients with myelofibrosis, in particular with primary myelofibrosis.

The purpose of this study is to investigate the safety, pharmacokinetics and preliminary efficacy of combination treatment of ruxolitinib with siremadlin in MF subjects. The study consists of three parts:

Part 1: Dose escalation and safety run-in (recommended Phase II dose confirmation)

Part 2: Selection

Part 3: Expansion

Purpose of Rationale:

Myelofibrosis (MF) is defined by progressive bone marrow (BM) fibrosis and a consecutive reduction of blood cells. The disruption of the medullary erythropoietic niche is the primary mechanism governing the bone marrow failure and anemia, which typify MF. Nearly 40% of MF patients have hemoglobin (Hb) levels <10 g/dL at diagnosis. Furthermore, anemia is the disease feature most consistently associated with poor prognosis in MF.

Ruxolitinib demonstrates improvements in splenomegaly and constitutional symptoms, however, does not improve anemia.

The purpose of this study is to investigate the safety, pharmacokinetics (PK) and preliminary efficacy of combinations treatment of ruxolitinib with novel compound siremadlin in MF subjects. This combination therapy may deliver transformational clinical benefits such as improvement of progression free survival (PFS) as a consequence of superior disease control or reduction of the malignant clone, associated with an improvement of cytopenia and in particular anemia, as well as improvement in quality of life (QoL) as captured by relevant patient reported outcomes measurements (PROs).

Key Inclusion Criteria:

Subjects have diagnosis of primary myelofibrosis (PMF) according to the 2016 World Health Organization (WHO) criteria, or diagnosis of postessential thrombocythemia (ET) (PET-MF) or post-polycythemia vera (PV) myelofibrosis (PPV-MF) according to the International Working Group for Myelofibrosis Research and Treatment (IWG-MRT) 2007 criteria;

Palpable spleen of at least 5 cm from the left costal margin (LCM) to the point of greatest splenic protrusion or enlarged spleen volume of at least 450 cm3 per MRI or CT scan at baseline (a MRI/CT scan up to 8 weeks prior to first dose of study treatment can be accepted).

Have been treated with ruxolitinib for at least 24 weeks prior to first dose of study treatment.

Are stable (no dose adjustments) on the prescribed ruxolitinib dose (between 5 and 25 mg twice a day (BID)) for ≥8 weeks prior to first dose of study treatment.

Hemoglobin <10 g/dL

Part 1: Platelet counts ≥75 000/μL

Part 2 and Part 3: Platelet counts ≥50 000/μL.

Key Exclusion Criteria

Not able to understand and to comply with study instructions and requirements.

Received any investigational agent for the treatment of MF (except ruxolitinib) within 30 days of first dose of study treatment or within 5 halflives of the study treatment, whichever is greater.

Peripheral blood blasts count of >10%.

Received a monoclonal antibody (Ab) or immunoglobulin-based agent within 1 year of screening, or has documented severe hypersensitivity reactions/immunogenicity (IG) to a prior biologic.

Splenic irradiation within 6 months prior to the first dose of study drug.

Received blood platelet transfusion within 28 days prior to first dose of study treatment.

Subjects with known TP53 mutation or deletion of TP53.

Primary Objectives:

To evaluate the preliminary efficacy of each novel ruxolitinib combination treatment arm (Parts 2 & 3)

To characterize the safety, tolerability, and recommended phase 2 dose (RP2D) of combination partner used with ruxolitinib (Part 1)

Primary Endponts:

Response rate (RR) for the composite endpoint (anemia improvement of 1.5 g/dL and no spleen volume progression and no symptom worsening) at the end of Cycle 6.

Incidence and severity of dose limiting toxicity (DLTs) within the first 2 treatment cycles in Part 1 of the study

Secondary Objectives:

To assess the proportion of subjects in each treatment arm who achieved an Hb improvement of g/dL or 1.5 g/dL (Parts 2 & 3).

To evaluate changes in symptoms of myelofibrosis in each treatment arm using MFSAF v4.0 and EORTC QLQ-C30 patient reported outcomes (PROs) (Parts 2 & 3).

To characterize the pharmacokinetic profile of ruxolitinib administered in combination with siremadlin (Parts 1, 2 & 3).

To evaluate the changes in spleen size in each treatment arm (Parts 2 & 3).

To evaluate the effect of ruxolitinib combination treatment in delaying progression of MF and estimate time to progression free survival (PFS) event (Parts 2 & 3).

To evaluate the effect on bone marrow fibrosis in each treatment arm (Parts 2 & 3).

To evaluate long-term safety and tolerability of ruxolitinib combination treatments (Parts 1, 2 & 3).

Secondary Endppoints:

Change in MFSAF v4.0 and EORTC QLQ-C30 from Baseline.

PK parameters (e.g., AUC, Cmax, Tmax) and concentration vs. time profiles of each investigational drug within combination regimens.

Change in spleen length (by palpation) from baseline.

Change in spleen volume (by MRI/CT) from baseline.

Estimate of progression free survival (PFS) where events are defined as follows:

Progressive splenomegaly as assessed by increasing spleen volume (by MRI/CT) of ≥25% from baseline. The progression date will be the date of MRI/CT assessment confirming spleen volume increase of ≥25% from baseline;

Accelerated phase defined by a circulating peripheral blood blast content of >10% but <20% confirmed after 2 weeks. The progression date will be the date of first increase in peripheral blood blast content of >10%;

Deteriorating cytopenia (dCP) independent from treatment defined for all patients by platelet count <35×10\hat{ }9/L or neutrophil count <0.75×10\hat{ }9/L that lasts for at least 4 weeks. The progression date will be the date of first decrease of platelets <35×10\hat{ }9/L or neutrophils <0.75×10\hat{ }9/L confirmed after 4 weeks;

Leukemic transformation defined by a peripheral blood blast content of ≥20% associated with an absolute blast count of ≥1×10\hat{ }9/L that lasts for at least 2 weeks or a bone marrow blast count of ≥20%. The progression date will be the date of first increase in peripheral blood blast content of ≥20% associated with an absolute blast count of ≥1×10\hat{ }9/L OR the date of the bone marrow blast count of 20%;

Death from any cause.

Proportion of subjects achieving improvement in bone marrow fibrosis of 1 grade from baseline Frequency, duration and severity of adverse events, abnormalities in vital signs and laboratory test values, including ECG data

Preliminary Results from Clinical Trials

PK data available from 7 patients treated with ruxolitinib receiving 10, 15 or 20 mg BID and from 6 patients treated with siremadlin receiving 20 mg QD. Dose reductions/interruptions were not taken into consideration for this preliminary PK analysis. Nominal times were used.

This study was not specifically designed to investigate the potential for PK drug interactions, however comparative assessment of historical data has been done. A PK DDI between ruxolitinib and siremadlin is unlikely or predicted to be low, based on PBPK analysis. A predicted <1.3-fold transient increase of ruxolitinib exposure (AUC and Cmax), through CYP3A4 inhibition may be observed.

Following dosing of ruxolitinib, absorption was fast and time to reach Cmax ranged from 0.5 to 2 h which was consistent with the known PK profile of this drug. Exposure was within the exposure range of values observed previously in MF patients receiving ruxolitinib as a single agent. Half-life was approximately 3-4 hours, little accumulation was observed upon repeated dosing.

Following dosing of siremadline, time to reach Cmax ranged from 2 to 8 h which was consistent with the known PK profile of this drug. Exposure was comparable to exposures observed previously in patients with solid and hematological malignancies receiving siremadlin as a single agent.

7 patients in cohort 1 were treated with 20 mg Siremadlin in combination with Ruxolitinib. There is no DLTs and no on treatment SAEs reported. 1 patient potentially not evaluable due to Siremadlin interruption within first 2 cycles of treatment. PK DDI between ruxolitinib and Siremadlin is unlikely or predicted to be low, based on PBPK analysis. As per statistical model the dose of Siremadlin can be increased to 30 mg or 40 mg D1-D5 in the next cohort.

1. An MDM2 inhibitor for use in the treatment of myelofibrosis (MF) in a patient.

2. The MDM2 inhibitor for use according to claim 1, wherein myelofibrosis comprises primary myelofibrosis (PMF), post-essential thrombocythemia myelofibrosis (PET-MF) and post-polycythemia vera myelofibrosis (PPV-MF).

3. The MDM2 inhibitor for use according to claim 1, wherein myelofibrosis is primary myelofibrosis (PMF).

4. The MDM2 inhibitor for use according to claim 1 wherein median survival time increases by at least 3 months.

5. The MDM2 inhibitor for use according to claim 1 wherein said patient completely responds to the treatment.

6. The MDM2 inhibitor for use according to claim 1, wherein said MF is newly diagnosed MF.

7. The MDM2 inhibitor for use according to claim 1, wherein MDM2 inhibitor, is administered in combination with at least one further active agent.

8. The MDM2 inhibitor for use according to claim 7, wherein the at least one further active agent is a JAK1/JAK2 inhibitor, a JAK2/FLT3 inhibitor, a JAK2^{V617F} inhibitor, a JAK2 inhibitor, JAK1 inhibitor or a JAK2/Src inhibitor, such as ruxolitinib, or a pharmaceutically acceptable salt thereof.

9. The MDM2 inhibitor for use according to claim 8, wherein ruxolitinib or a pharmaceutically acceptable salt thereof, is administered in an amount of from 5 mg twice daily to 25 mg twice daily, such as 5 mg twice daily, 10 mg twice daily, 15 mg twice daily, 20 mg twice daily or 25 mg twice daily.

10. The MDM2 inhibitor for use according to claim 1, wherein said MDM2 inhibitor is siremadlin or pharmaceutical acceptable salt thereof.
